(19) United States
(12) Patent Application Publication
(10) Pub. No.: US 2011/0294677 A1
(43) Pub. Date: Dec. 1, 2011

(54) MICROFLUIDIC SYSTEMS COMPRISING A RUBBER MATERIAL SUBSTRATE

(75) Inventors: Lucas Johannes Anna Maria Beckers, Eindhoven (NL); Remco Virgil Woen, Eindhoven (NL); Joost Hubert Maas, Eindhoven (NL)

(73) Assignee: KONINKLIJKE PHILIPS ELECTRONICS N.V., EINDHOVEN (NL)

(21) Appl. No.: 13/148,070

(22) PCT Filed: Feb. 18, 2010
(86) PCT No.: PCT/IB2010/050719

§ 371 Date: Aug. 5, 2011

(30) Foreign Application Priority Data
Feb. 20, 2009 (EP) 09153280.4

(51) Int. Cl. C40B 30/00; A61M 5/00; C12Q 1/00; G01N 33/00; B01L 3/00; C12M 1/00

(52) U.S. Cl. 506/7; 422/502; 435/283.1; 435/4; 436/86; 604/93.01

(57) ABSTRACT

The invention relates to a novel microfluidic system having a substrate based on rubber material having polar side groups which are linked to the rubber polymer backbone via a spacer. By doing so, a transport of water-based fluids such as blood, saliva etc. will occur by capillary forces.

### FIELD OF THE INVENTION

The present invention is directed to microfluidic systems, especially to microfluidic systems for use in the detection of analytes in fluids, especially body fluids.

### BACKGROUND OF THE INVENTION

This invention relates to a microfluidic device for molecular diagnostic applications such as labs-on-a-chip or micro total analysis systems, to a disposable cartridge comprising said micro fluidic device and to the uses thereof. The microfluidic device according to the present invention is preferably used in molecular diagnostics.

The biotechnology sector has directed substantial effort toward developing miniaturized microfluidic devices, often termed labs-on-a-chip (LOC) or micro total analysis systems, (micro-TAS), for sample manipulation and analysis. These systems are used for detection and analysis of specific bio-molecules, such as nucleic acids and proteins.

In general, micro-system devices contain fluidic, electrical and mechanical functions, comprising pumps, valves, mixers, heaters, and sensors such as optical-, magnetic- and/or electrical sensors. A typical molecular diagnostics assay includes process steps such as cell lysis, washing, amplification by PCR, and/or detection.

Integrated microfluidic devices need to combine a number of functions, like filtering, mixing, fluid actuation, valving, heating, cooling, and optical, electrical or magnetic detection, on a single template. Following a modular concept the different functions can be realized on separate functional substrates, like silicon or glass. The functions need to be assembled with a microfluidic channel system, which is typically made of plastic. With small channel geometries this way of integration becomes a very challenging process. The interfaces between the substrates and the channel plate need to be very smooth and accurate, and the channel geometries need to be reproducible, while the functional substrates should have a minimum footprint for cost and raw material efficiency. Especially with functions which need a fluidic as well as an electric interface, the separation of the wet interface is critical. Bonding techniques must be compatible with the biochemical reagents and surface treatments present on the functional substrates.

US-A1 2003/0057391, incorporated by reference, discloses a low power integrated pumping and valving array which provide a revolutionary approach for performing pumping and valving operations in micro fabricated fluidic systems for applications such as medical diagnostic microchips. This approach integrates a lower power, high-pressure source with a polymer, ceramic, or metal plug enclosed within a micro channel, analogous to a micro syringe. When the pressure source is activated, the polymer plug slides within the micro channel, pumping the fluid on the opposite side of the plug without allowing fluid to leak around the plug. The plugs also can serve as micro valves.

However, the pump system of US-A1 2003/0057391 does not provide a sufficient small dead volume and does not provide an optimized fast fluid transport. Further, the plugs must have a positive fitting to avoid sample fluid leakage thus the low power integrated pumping and valving arrays cannot be provided at low vertical range of manufacture.

In the last decade, considerable research efforts have been made to develop microfluidic system devices in order to integrate more functions while at the same time reducing sample volumes.

Despite this effort, there is still a need for microfluidic system devices, such as microfluidic bio chips, often termed Bio Flips, LOCs and micro-TASs, to overcome at least one drawback of the prior art mentioned above. Further, there is a need to develop technologies that lead to total integration of peripheral functions onto single microchips, including innovative low power/pressure sources for on-chip fluidic manifolds that allow analyzing samples in small volumes of liquid as well as providing more economical use of reagents and samples.

### SUMMARY OF THE INVENTION

It is an object of the present invention to provide a microfluidic system which allows a less sophisticated set-up according to the prior art solution, and which can for many applications, be manufactured and employed easily.

This object is solved by the rubber material recited in claim 1 of the present invention. Accordingly, a micro fluidic system is provided, comprising a substrate having a surface with at least one micro channel structure thereon whereby at least a part of said substrate comprises a rubber material which comprises polar side groups whereby each of the polar side groups is linked with the polymer chain of said rubber material via a linker comprising at least 6 atoms.

Such a microfluidic system has shown a wide range of applications within the present invention to have at least one of the following advantages:

the rubber material can be produced and handled in a bulk-scale fashion;

the rubber material is usable in injection-molding techniques;

the rubber material can be made out of readily available precursor materials, usually without the requirement of sophisticated production steps;

due to the polar side groups, an active transport of water-based fluids, (e.g., blood, saliva, etc.), is for many applications, no longer needed or only to a small extent, for example, when valves are needed. Due to capillary forces and the hydrophilic properties of the rubber material, the sample fluid will flow through the micro channels “independently”. This allows the set-up of much more sophisticated microfluidic systems due to the non-necessity of setting up valves, pressure chambers, etc.

for many uses of the invention the application of coatings used for sealing the micro fluidic devices is no longer necessary;

for many applications of the invention, bonding requirements with glue or heat sealing, (e.g., ultrasound or laser welding, and the like), are no longer needed because the elastomer properties of the rubber material substrate automatically closes, or seals itself against the adjacent surface(s) of the microfluidic device.

In the sense of the present invention, the term “substrate” especially includes and/or means a flat part with a (micro) fluidic pattern.

In the sense of the present invention the term “micro channel” especially includes and/or means a channel with a width of 1000 to 1 micron.

It should be noted that in the context of the present invention it is not necessary that the entire substrate is made out of the inventive rubber material, although this is ad libitum for the skilled person in the art and a preferred embodiment of the present invention. In order to achieve most (or at least some) of the advantages of the present invention it might be possible that only a part of the substrate comprises said rubber material. If this is so, then it is preferred that at least a part of the substrate that forms a micro channel comprises the inventive rubber material.

In the sense of the present invention, the term “rubber material” especially includes and/or means an elastomeric material. Examples of suitable materials which may be used in the context of this invention are:

NBR nitrile rubbers in the form of butadiene-acrylonitrile co- or terpolymers,

HNBR partially or fully hydrogenated nitrile rubbers in the form of hydrogenated butadiene-acrylonitrile co- or terpolymers,

XNBR carboxylated nitrile rubbers,

HXNBR partially or fully hydrogenated carboxylated nitrile rubbers,

EVM ethylene-vinyl acetate copolymers,

EPDM ethylene-propylene-diene copolymers,

ESBR styrene-butadiene copolymers,

CR polychloroprene,

BR polybutadiene,

ACM acrylate rubber,

FKM fluororubber,

IIR isobutylene-isoprene copolymers, usually with isoprene contents of from 0.5 to 0% by weight,

BIIR brominated isobutylene-isoprene copolymers, usually with bromine contents of from 0.1 to 10% by weight,

CIIR chlorinated isobutylene-isoprene copolymers, usually with chlorine contents of from 0.1 to 10% by weight,

ABR butadiene-C-4-alkyl acrylate copolymers,

IR polyisoprene,

X-SBR carboxylated styrene-butadiene copolymers

EAM ethylene-acrylate copolymers,

CO and

ECO epichlorohydrin rubbers,

Q silicone rubbers,

AU polyester urethane polymers,

EU polyether urethane polymers,

ENR epoxidized natural rubber or a mixture thereof.

For the purposes of this application, nitrile rubbers, also known by the abbreviated term NBR are co- or terpolymers which contain repeat units and at least one conjugated diene, of at least one alpha, beta-unsaturated nitrile and, if appropriate, one or more other copolymerizable monomers.

The conjugated diene can be of any type. It is preferable to use C4-C6 conjugated dienes. Particular preference is given to 1,3-butadiene, isoprene, 2,3-dimethylbutadiene, piperylene or a mixture thereof. Particular preference is given to 1,3-butadiene and isoprene or a mixture thereof. The C4-C6 conjugated diene, 1,3-butadiene, is very particularly preferred.

The alpha, beta-unsaturated nitrile used can comprise any known alpha, beta-unsaturated nitrile, and preference is given to C3-C5 alpha, beta-unsaturated nitriles, such as acrylonitrile, methacrylonitrile, ethacrylonitrile or a mixture of these. Acrylonitrile is particularly preferred.

Particularly preferred nitrile rubber is provided by a copolymer based on the monomers acrylonitrile and 1,3-butadiene.

### Hydrogenated Nitrite Rubbers (HNBR)

For the purposes of this application, hydrogenated nitrile rubbers (HNBR) are co- or terpolymers which contain repeat units of at least one conjugated diene, of at least one alpha, beta-unsaturated nitrile and, if appropriate, of one or more copolymerizable monomers, and in which the C═C double bonds of the diene units incorporated into the polymer have been hydrogenated to some extent.

The conjugated diene can be of any type. It is preferable to use C4-C6 conjugated dienes. Particular preference is given to 1,3-butadiene, isoprene, 2,3-dimethylbutadiene, piperylene or a mixture thereof. Particular preference is given to 1,3-butadiene and isoprene or a mixture thereof. The C4-C6 conjugated diene, 1,3-butadiene, is very particularly preferred.

The alpha, beta-unsaturated nitrile used can comprise any known alpha, beta-unsaturated nitrile, and preference is given to C3-C5 alpha, beta-unsaturated nitrites, such as acrylonitrile, methacrylonitrile, ethacrylonitrile or a mixture of these. Acrylonitrile is particularly preferred.

Alongside the conjugated diene and the alpha, beta-unsaturated nitrile, it is also possible to use one or more other monomers known to the person skilled in the art, examples being alpha, beta-unsaturated mono- or dicarboxylic acids, or their esters or amides. Preferred alpha, beta-unsaturated mono- or dicarboxylic acids here are fumaric acid, maleic acid, acrylic acid and methacrylic acid. Preferred esters used of the alpha, beta-unsaturated carboxylic acids are their alkyl esters and alkoxyalkyl esters. Particularly preferred esters of the alpha, beta-unsaturated carboxylic acids are methyl acrylate, ethyl acrylate, butyl acrylate, butyl methacrylate, 2-ethylhexyl acrylate, 2-ethylhexyl methacrylate and octyl acrylate.

If the hydrogenated nitrile rubber has carboxy groups, the term HXNBR is also used.

Suitable rubbers may comprise ethylene-vinyl acetate (EVM) copolymers based on ethylene and vinyl acetate as monomers.

Ethylene-vinyl acetate copolymers which can be used for the purposes of the invention are commercially available, e.g., as products from the product range with trade names Levapren® and Levamelt® from Lanxess Deutschland GmbH, or else can be prepared by the familiar methods known to the person skilled in the art.

### EPDM Rubbers

EPDM rubbers are polymers prepared via terpolymerization of ethylene and of relatively large proportions of propylene, and also of a few % by weight of a third monomer having diene structure. The diene monomer here provides the double bonds needed for any subsequent sulphur vulcanization. Diene monomers mainly used are cis,cis-1,5-cyclooctadiene (COD), exo-dicyclopentadiene (DCP), endo-dicyclopentadiene (EDCP), 1,4-hexadiene (HX) and also 5-ethylidene-2-norbornene (ENB).

EPDM rubbers which can be used for the purposes of the invention are commercially available, e.g., as products from the product series with the trade name Buna EP® from Lanxess Buna GmbH, or else can be prepared by the methods familiar to the person skilled in the art.

### Emulsions Styrene-Butadiene Rubber (ESBR)

This material involves copolymers composed of the monomers styrene and butadiene. The materials are prepared via emulsion polymerization in water, initiated by redox initiators at low temperatures or at relatively high temperatures by persulphates. Latices are obtained and are used as they stand or else worked up to give solid rubber. The molar masses of ESBR are in the range from about 250 000 to 800 000 g/mol.

Emulsion styrene-butadiene rubbers which can be used for the purposes of the invention are commercially available, e.g., as products from the product range with trade names Krynol® and Krylene® from Lanxess Deutschland GmbH, or else can be prepared by methods familiar to the person skilled in the art.

### Chloroprene Rubber (CR)

Chloroprene rubbers (CR) involve polymers based on chloroprene (chloro-1,3-butadiene), these being prepared industrially via emulsion polymerization. Preparation of CR can use not only chloroprene but also one or more other monomers.

Chloroprene rubbers (CR) which can be used for the purposes of the invention are available commercially, e.g., as products from the product range with the trade name Baypren® from Lanxess Deutschland GmbH, or else can be prepared by methods familiar to the person skilled in the art.

### Polybutadiene Rubbers (BR)

These involve poly(1,3-butadiene), a polymer based on 1,3-butadiene.

### Acrylate Rubbers (ACM)

Acrylate rubbers involve copolymers prepared by a free-radical route in emulsion and composed of ethyl acrylate with other acrylates, such as butyl acrylate, 2-alkoxyethyl acrylates or other acrylates having, incorporated into the polymer, small proportions of groups which are active in vulcanization.

ACM rubbers which can be used for the purposes of the invention are commercially available, e.g., as products from the product range with trade names Hy Temp®/Nipol® AR from Zeon Chemicals, or else can be prepared by methods familiar to the person skilled in the art.

### Fluororubbers (FKM)

These involve copolymers, prepared by a free-radical route in emulsion, of fluorinated ethylene monomers with fluorinated vinyl monomers and also, if appropriate, with other monomers, where these supply groups that are active in vulcanization.

FKM rubbers which can be used for the purposes of the invention are commercially available, e.g., as products from the product range with the trade name Viton® from DuPont des Nemours, or else can be prepared by methods familiar to the person skilled in the art.

### IIR and Halo IIR (BIIR and CIIR)

Butyl rubbers (IIR) are a copolymer composed of isobutene and of small proportions of isoprene. They are prepared by a cationic polymerization process. Halobutyl rubbers (BIIR and CIIR) are prepared therefrom via reaction with elemental chlorine or bromine.

Butyl rubbers and halobutyl rubbers which can be used for the purposes of the invention are commercially available, e.g., as products from the product range with trade names Lanxess Butyl and Lanxess Chlorobutyl and, respectively, Lanxess Bromobutyl from Lanxess Deutschland GmbH, or else can be prepared by methods familiar to the person skilled in the art.

### Polysiloxanes

Especially preferred rubber materials in the context of the present invention comprise polysiloxanes. Especially preferred are derivatives of Polydialkylsiloxanes, Polydiarylsiloxanes and/or Polydialkyl/arylsiloxanes, especially derivatives of Polydimethylsiloxane. Preferred chain lengths are from 1000 to 10,000, preferably between 6000 and 1000 Si—O-units.

It should be noted that the rubber material may be present as a uniform material or a block or graft polymer.

The term “polar” side group especially means and/or includes a chemical moiety which has a δ⁺ and δ⁻. Polar side groups may be ionic, however, also non-ionic side groups may be used within the present invention. Preferred non-ionic side groups comprise (although this is not limiting) hydroxy, amide, ester.

The term “ionic side” group especially means and/or includes that the rubber material comprises a chemical moiety which is charged when the rubber material is used. Preferably the chemical moiety is charged at neutral pH. Preferred ionic side groups comprise at least one moiety selected from of the group —SO₃⁻, —OPO₄²⁻, —PO₃²⁻, —OSO₂⁻, —CO₂⁻, —NR₁R₂R₃⁺, —PR₁R₂R₃⁺. Preferred counter-ions comprise alkali metal ions, earth alkali metal ions, H⁺, NH₄⁺ or mixtures thereof (for the negative charged polar side groups) or halogenides, OH⁻, BF₄⁻ or mixtures thereof (for the positive charged polar side groups).

Although this is a preferred embodiment of the present invention, the polar side group does not need to form an “end group”; it may also be present as a side group, e.g., in an alkyl chain (at a secondary carbon).

The term “linker comprising at least 6 atoms” especially means and/or includes a polar side group that is spaced along the polymer chain of the rubber via a chain (e.g., a carbon chain or a substituted carbon chain like a polyethoxide chain).

It should be noted that the length of the linkers do not necessarily have to be uniform across the rubber material although it is preferred that ≧80%, more preferred ≧90% and most preferred ≧95% of all linkers have the same length.

The term “polymer chain” is to be understood in its broadest sense and also includes that the rubber material may be crosslinked. Therefore the term “polymer chain” may also include a “polymer network”.

Preferably each of the polar side groups is linked with the polymer chain of said rubber material via a linker comprising at least 8 atoms, more preferably at least 10 atoms, yet more preferred 12 atoms and most preferred at least 14 atoms.

According to a preferred embodiment of the present invention, the content of said polar side groups is set so that the wetting angle of water towards the rubber is ≦80°, preferably ≦70°, more preferably ≦55°. When the silicon rubber is modified with 15 w % sodium alkene (C14-C16) sulfonate, (SAS), the wetting angle of water to its surface is 70-75°. And when the silicon rubber is modified with 20 w % SAS (C14-C16) the wetting angle of water to its surface is 50-55°.

According to a preferred embodiment of the present invention, the content of said polar side groups is ≧0.01 and ≦1 mol per 100 g rubber material.

This has been shown to be advantageous for many applications within the present invention. If the content of said polar side groups is too low the rubber material will show only water transportation. On the other hand if the content of the polar side groups is too high, the rubber material will lose many of its advantageous features (since it will slowly turn into a detergent).

It is especially preferred that the content of said polar side groups is ≧0.025 and ≦0.8, more preferred ≧0.05 and ≦0.3 and most preferred ≧0.075 and ≦0.2 moles per 100 g rubber.

According to a preferred embodiment of the present invention, the polar side groups are linked with the polymer chain of said rubber material via a carbon chain.

According to a preferred embodiment, the rubber material comprises at least one material comprising the following structural unit:

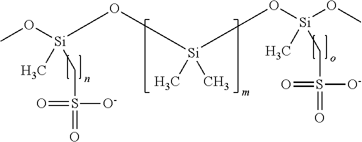

with n,o independently from each other being 6 or larger.

This structural unit may be made by radical addition of \acute{ω}-alkenylsulfonic acids to vinyl-substituted siloxaneunits present in the polysiloxane chain.

According to a preferred embodiment, the rubber material has a tensile strength from ≧2 and ≦8, preferably ≧3 and ≦6 and most preferred ≧4 and ≦5 MPa.

According to a preferred embodiment, the rubber material has an elongation from ≧100% and ≦800%, preferably ≧300% and ≦600% and most preferred ≧400% and ≦500%.

According to a preferred embodiment of the present invention, the rubber material is made by a process comprising the step of radical addition of a suitable rubber precursor monomer with an ionic precursor material.

This has proven itself in practice since by doing so the inventive rubber material may be produced easily. The step of radical addition may e.g., be performed by radical dimerization of alkene moieties or by any other known bonding technique in the field. It may be performed by a radical initiator (e.g., peroxides, AIBN, tin organyls etc.) or by UV-light.

According to a preferred embodiment of the present invention, the rubber material is made by a process comprising the step of radical addition of a suitable rubber precursor monomer with an ionic precursor material at a temperature of ≧80° C.

A microfluidic device according to the present invention may be of use in a broad variety of systems and/or applications, amongst them one or more of the following:

biosensors used for molecular diagnostics;

rapid and sensitive detection of proteins and nucleic acids in complex; biological mixtures such as e.g., blood or saliva;

high throughput screening devices for chemistry, pharmaceuticals or molecular biology;

testing devices e.g., for nucleic acids or proteins e.g., in criminology, for on-site testing (in a hospital), for diagnostics in centralized laboratories or in scientific research;

tools for nucleic acid or protein diagnostics for cardiology, infectious disease and oncology, food, and environmental diagnostics;

tools for combinatorial chemistry;

analysis devices;

nano- and micro-fluidic devices;

fluid pumping devices;

rug release and drug delivery systems (in particular transdermal and implantable drug delivery devices).

The aforementioned components, as well as the claimed components and the components to be used in accordance with the invention in the described embodiments, are not subject to any special exceptions with respect to their size, shape, material selection and technical concept such that the selection criteria known in the pertinent field can be applied without limitations.

### BRIEF DESCRIPTION OF THE DRAWINGS

Additional details, features, characteristics and advantages of the object of the invention are disclosed in the subclaims, the figures and the following description of the respective figures and examples, which—in an exemplary fashion—show several embodiments and examples of a rubber material and a microfluidic device according to the present invention.

FIG. 1 shows a picture of a mold for the structuring of a rubber material according to a first embodiment of the present invention.

FIG. 2 shows a detailed view of FIG. 1.

FIG. 3 shows a detailed view of a microstructure using a rubber material (which was structured by using the mold in FIGS. 1 and 2).

FIG. 4 shows the microstructure after the injection of colored water.

FIG. 5 shows the same microstructure as FIG. 4 after a few seconds; and

FIG. 6 shows the same microstructure as FIGS. 4 and 5 after a few more seconds.

FIG. 7 shows the experimental results of EXAMPLE 1 which is a comparison of the tensile strength and elongation of non-modified silicon rubber, made according to the manufacturer's instructions versus the modified rubber material of the invention.

### DETAILED DESCRIPTION OF THE EMBODIMENTS

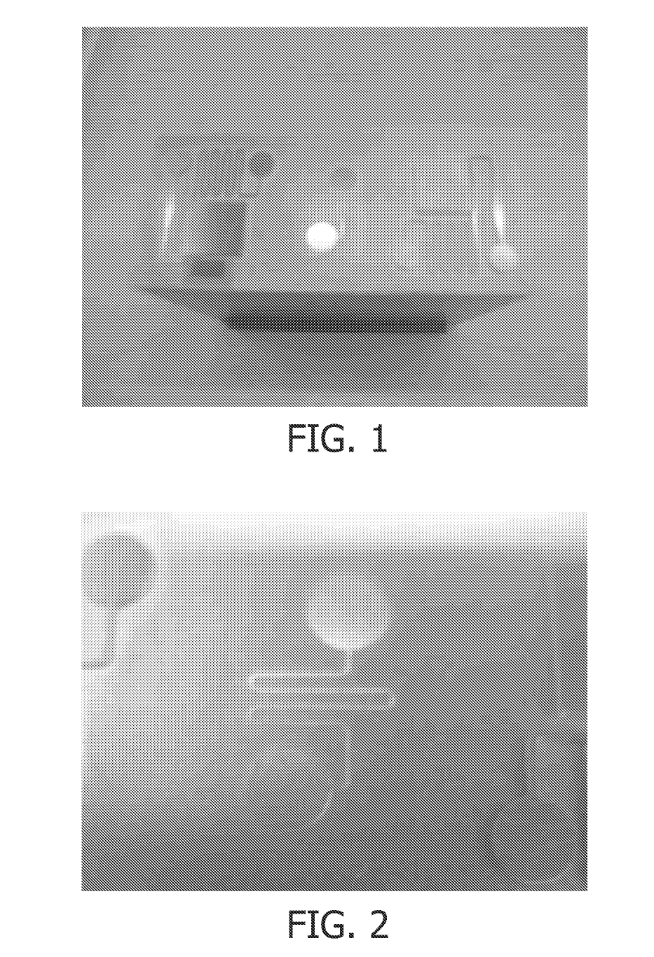

FIG. 1 shows a picture of a mold for the structuring of a rubber material according to a first embodiment of the present invention. The mold as such is prior art and any techniques used in the field can be used. FIG. 2 shows a detailed view of FIG. 1 (where the channel structure to illustrate the advantageous use of the inventive rubber material can better be seen).

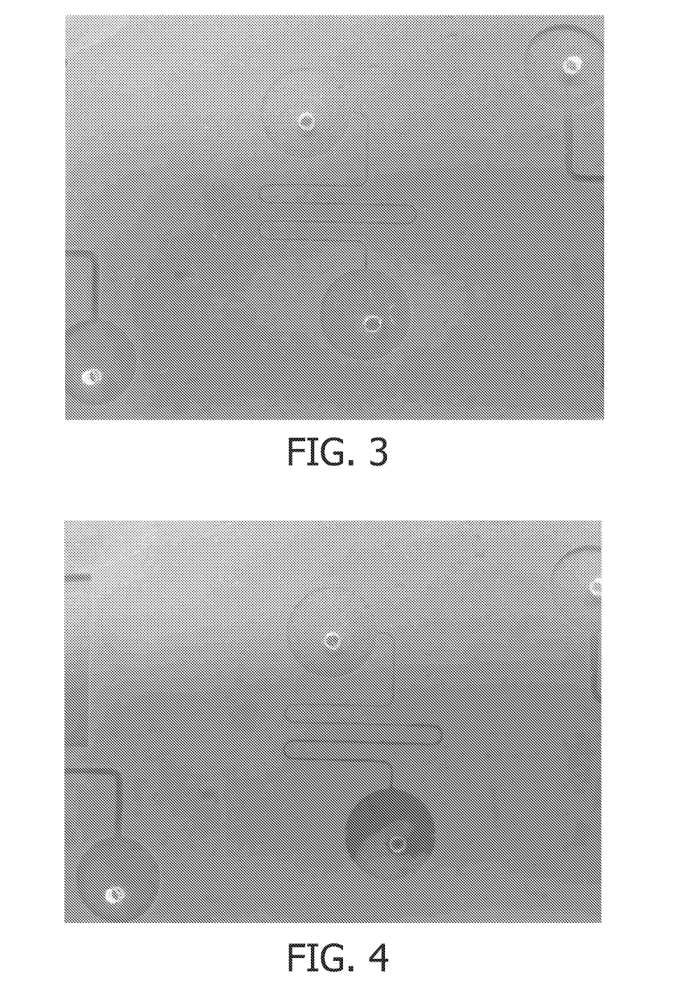

FIG. 3 shows a detailed view of a microstructure using a rubber material (which was structured by using the mold in FIGS. 1 and 2). The microstructure comprises essentially two parts, i.e., a rubber material according to a first embodiment of the present invention and a glass plate.

The rubber material used in this microstructure was made by altering the production procedure of the commercially available silicon material Elastosil® LR 3003/60 US from Wacker Silicones. Reference is made to the Product data sheet Version No. 4.00. Elastosil® comprises two silicone components named “Components A and B” on Wacker Silicones' Technical data sheet, Version 1.1, also referred to as ELASTOSIL® LR 3003/60 A and ELASTOSIL® LR 3003/60 B on Wacker Silicones' corresponding Material Safety Data Sheets, and “parts A & B” on Wacker Silicones' Product data sheet, Version 4.00, out of which the rubber material is in situ made before using. For the sake of brevity, these two components hereinafter are referred to as “component A” and “component B”.

The manufacturing procedure of Elastosil® LR 3003/60 US was altered as follows.

Silicone component A, containing vinyl groups on the Siloxane chain, with platinum catalyst, was high speed mixed with Sodium alkene (C14-C16) sulfonate. After mixing, the mixture was heated up to 120 degrees Celsius and mixed again.

After cooling down at room temperature, to room temperature, Silicone component B was added. Component B comprises Hydro-Silicon bondings which function as a cross-linker. The two components are high speed mixed again. The mixture was prepared in a cartouche which could be used to feed the injection molding equipment. The cartouche was held under pressure for constant feeding.

Injection molding occurred in a mold for shaping the fluidic devices (or fluidic membranes) at a mold temperature of 180 degrees Celsius. Injection/de-molding cycles were done in 25 seconds.

The wetting angle of the inventive rubber material was approx. 55°; the content of the sulfonate groups per 100 g rubber was around 0.11 mole. Tensile strength was 4.5 Mpa and elongation approximately 450%.

The glass plate was stuck to the rubber substrate simply due to the adhesive and sticky properties of the inventive rubber material. No “glue” or adhesives of any type are necessary.

FIG. 4 shows the microstructure after the injection of blue colored water (in the “lower” reservoir). The color is simply for illustration purposes; any water-based liquid could be used.

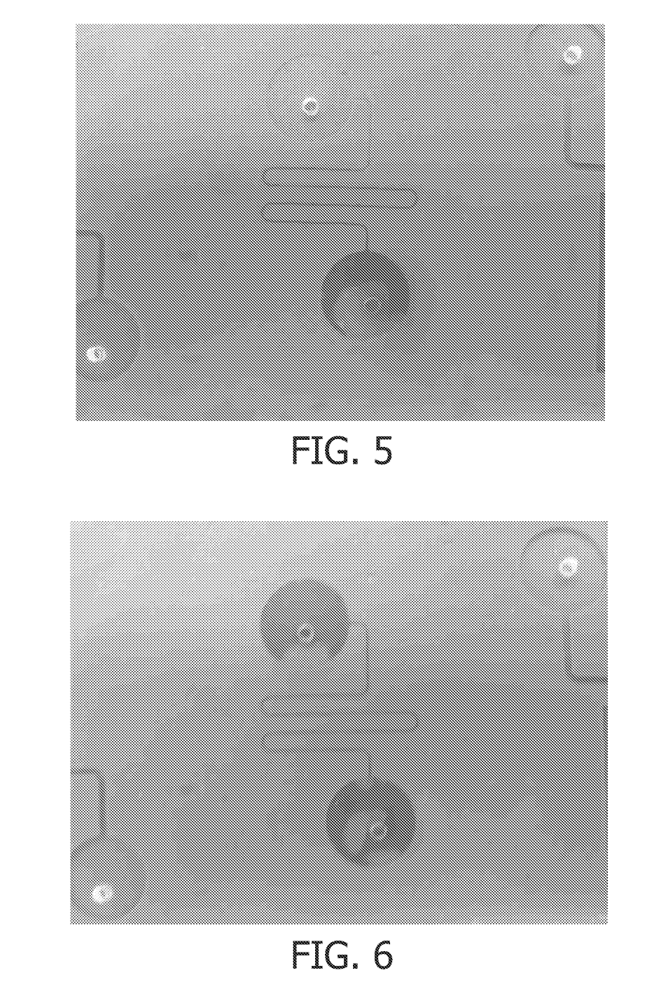

It can be seen that the water flows, (due to capillary forces), through the microstructure. FIG. 5 shows the same microstructure after a few seconds; and FIG. 6 shows the same microstructure further after a few seconds. After approximately 10 seconds, the water has reached the “upper” reservoir.

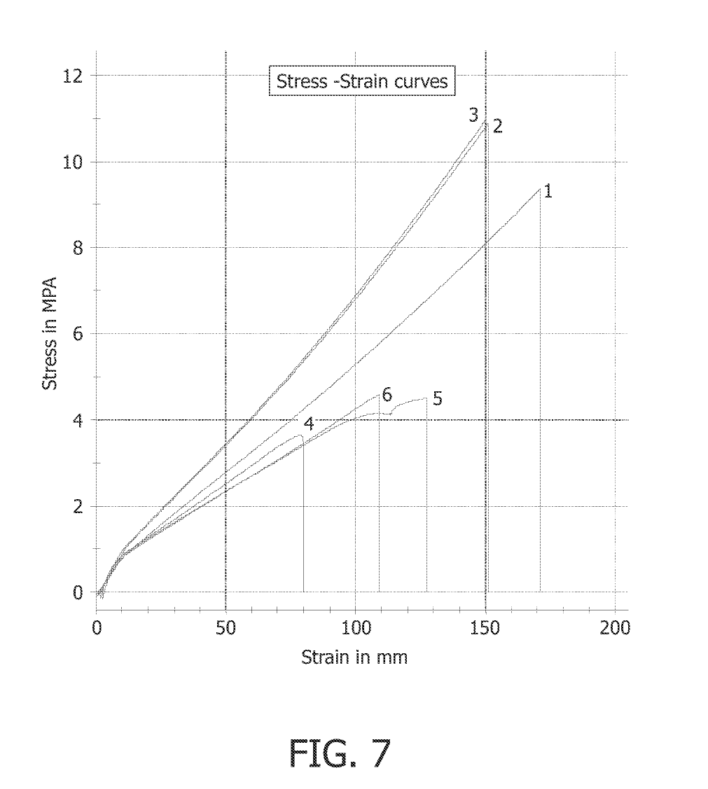

FIG. 7 shows the experimental results of EXAMPLE 1, (shown below) which is a comparison of the tensile strength and elongation of non-modified silicon rubber, made according to the manufacturer's instructions, and a modified silicon rubber comprising SAS.

### Example 1

The rubber used in this example was ELASTOSIL® LR 3003/60 US. The non-modified silicon rubber samples and the modified silicon rubber plus SAS were injection molded. The tensile strength and the elongation of 2×3 samples were measured by means of the Zwick draw bench type 1474. The first set of samples labeled curves 1, 2 and 3 in FIG. 7 are the non-modified silicon rubber and the second set of curves, 4, 5 and 6 of FIG. 7, are the modified silicon rubber+SAS. Curves 4, 5 and 6 representing the modified silicon rubber of
the invention comprises 15% mass-percentage of SAS. The Zwick draw bench type 1474 with a 2 kN force cell was used to perform the stress strain measurements. Line clamps were used to hold the modified and non-modified silicon rubber samples in place. The results of the tests are shown in FIG. 7. The most important settings are listed below:

| | |
|---|---|
| Load cell | 2 kN |
| Extensometer (path) | Crosshead |
| Specimen holder | 8106 |
| Machine data | 1474 |
| Test type | Tensile |
| E-Modulus speed | 0.03 1/s |
| Begin E-Modulus determination | 3N |
| End E-Modulus determination | 6N |
| Test speed | 0.03 1/s |
| Test environment name | DrukHyst20N.ZPV |

TABLE 1

| # | Sample Type | Rm [Mpa] | Comments |
|---|---|---|---|
| 1 | RYH0, LVDR | 9.37 | - Ar = ca. 600%, Lgrips = 17 mm |
| 2 | RYH0, LVDR | 10.96 | - Ar = ca. 600%, Lgrips = 10 mm |
| 3 | RYH0, LVDR | 10.89 | - Ar = ca. 600%, Lgrips = 10 mm |
| 4 | AYH0, SAS, LVDR | 3.64 | - Ar = ca. 420%, sample slipped |
| 5 | AYH0, SAS, LVDR | 4.49 | - Ar = ca. 450%, Lgrips = 10 mm |
| 6 | AYH0, SAS, LVDR | 4.59 | - Ar = ca. 450%, Lgrips = 10 mm |

Table Legend: R: Reference; Y: Youngs modulus; H; High humidity; 0: 0 weeks; A; Modified silicon rubber sample comprising SAS; LVDR; type elastomer; Ar means elongation of the rubber; ca. means circa.

Table 1 above shows the stress strain results up to rupture of the two different sample sets (Specimen No.'s 1, 2 and 3 were the non-modified silicon rubber made according to the manufacturer's instructions and Specimen No.'s 4, 5 and 6 were the silicon rubber of the invention comprising 15% SAS. Curves 2 and 3 coincide very well, whereas curve 1 does not. This is due to the fact that for Specimen No. 1 the clamping was further apart than for the other specimens of the set. The curves for Specimen no.'s 4, 5 and 6 do not coincide, but there was a reason for this difference. Specimen No. 4 slipped though the clamping, causing the measurement to prematurely end. Therefore, the elongation result of curve 4 was smaller than for curve 5 and curve 6. Noticeable in FIG. 7, Curve 5 has a small dimple around 4 MPa. This is due to slipping of Specimen No. 5. When the slipping occurred the clamps were tightened, causing the dimple and the measuring resumed. FIG. 7 and Table 1 above show the modified silicon rubber+SAS of the invention has a tensile strength in the order of 4.5 MPa and an elongation in the order of 450%. It is important to note, and a surprise to the inventors that the elastomer properties of the modified silicon rubber plus the polar side groups did not change significantly, i.e., the elastomer properties were maintained relative to the non-modified silicon rubber material.

The elastomer properties, the wetting angles in the range of 50-75° and sticky properties of this newly modified silicon rubber material comprising polar side groups make it very suitable to combine with glass to form fluidic devices with autonomous flow behavior. Furthermore, it's been found that the hydrophilic properties of this new rubber material maintains even after laying in the open air over a prolonged period of time.

The particular combinations of elements and features in the above detailed embodiments are exemplary only; the interchanging and substitution of these teachings with other teachings in this and the patents/applications incorporated by reference are also expressly contemplated. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended as limiting. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage. The invention's scope is defined in the following claims and the equivalents thereto. Furthermore, reference signs used in the description and claims do not limit the scope of the invention as claimed.

1. Microfluidic system, comprising a substrate having a surface with at least one micro channel structure thereon whereby at least a part of said substrate comprises a rubber material which comprises polar side groups whereby each of the polar side groups is linked with the polymer chain of said rubber material via a linker comprising at least 6 atoms.

2. Microfluidic system according to claim 1, whereby the content of said polar side groups is set so that the wetting angle of water towards the rubber is ≦80°, preferably ≦70°, or more preferably ≦55°.

3. Microfluidic system according to claim 1, whereby said polar side groups in said rubber material comprise at least one moiety selected out of the group —SO₃⁻, —OPO₄²⁻, —PO₃²⁻, —OSO₂⁻, —CO₂⁻, —NR₁R₂R₃⁺.

4. Microfluidic system according to claim 1, whereby the content of said polar side groups is ≧0.01 and ≦1 mol per 100 g rubber material.

5. Microfluidic system according to claim 1, whereby the rubber material comprises at least one material comprising the following structural unit: 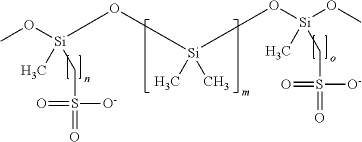
with n,o independently from each other being 6 or larger.

6. Microfluidic system according to, whereby the rubber material has a tensile strength from ≧2 and ≦8, preferably ≧3 and ≦6 most preferred ≧4 and ≦5 MPa.

7. Microfluidic system according to, whereby the rubber material has an elongation from ≧100% and ≦800%, preferably ≧300% and ≦600% and most preferred ≧400% and ≦500%.

8. Use of a Microfluidic system according to claim 1 as and/or with
biosensors used for molecular diagnostics
rapid and sensitive detection of proteins and nucleic acids in complex biological mixtures such as e.g., blood or saliva
high throughput screening devices for chemistry, pharmaceuticals or molecular biology
testing devices e.g., for nucleic acids or proteins e.g., in criminology, for on-site testing (in a hospital), for diagnostics in centralized laboratories or in scientific research
tools for nucleic acid or protein diagnostics for cardiology, infectious disease and oncology, food, and environmental diagnostics
tools for combinatorial chemistry
analysis devices
nano- and micro-fluidic devices
fluid pumping devices
drug release and drug delivery systems (in particular transdermal and implantable drug delivery devices).
